(19) United States
(12) Patent Application Publication
(10) Pub. No.: US 2011/0186048 A1
(43) Pub. Date: Aug. 4, 2011

(54) CONNECTION INTERFACE BETWEEN A BREATHING ASSISTANCE UNIT AND A PERIPHERAL DEVICE

(76) Inventors: Benjamin Wilson Casse, Auckland (NZ); Andrew Paul Maxwell Salmon, Auckland (NZ)

(21) Appl. No.: 13/016,781

(22) Filed: Jan. 28, 2011

Related U.S. Application Data
(63) Continuation of application No. PCT/NZ2009/000151, filed on Jul. 29, 2009.
(60) Provisional application No. 61/084,322, filed on Jul. 29, 2008.

(51) Int. Cl. A61M 16/16

(52) U.S. Cl. 128/203.26

(57) ABSTRACT

A removable peripheral device and a breathing assistance unit form part of a system for providing pressurized heated humidified air at to a user. The removable peripheral device and the breathing assistance unit are mutually adapted to be removably connectable to, and in electrical communication with, each other via a connection between the removable peripheral device and the breathing assistance unit. The removable peripheral device comprises a housing and a first connector extending from an end of the housing. The housing has a larger cross-section than the connector. The breathing assistance unit comprises an exterior casing and a second connector mounted within the casing and accessible from outside of the casing. An interfacing portion, the second connector and the housing formed so at least a portion of the housing adjacent the first connector end interfaces with the interfacing portion to connect the removable peripheral device to the breathing assistance unit.

### CROSS-REFERENCE TO RELATED APPLICATIONS

This application is a continuation under 35 U.S.C. §120 of PCT International Application No. PCT/NZ2009/000151, filed on Jul. 29, 2009, which claims priority to U.S. Provisional Application No. 61/084,322, filed Jul. 29, 2008, each of which is incorporated by reference in its entirety.

### BACKGROUND OF THE INVENTION

1. Field of the Invention

The present invention relates to a keyed interfacing arrangement between a peripheral device and a host device when connecting the peripheral device to the host device in an electrical or data communicating connection. More specifically, the present invention relates to the interface between a removable memory device and the host device.

2. Description of the Related Art

In certain circumstances, it may be desirable for a connector interface between two devices, such as a host device and a peripheral device, to be “keyed” in some way so that the host device is capable of recognizing or selectively accepting the connection of the peripheral device.

U.S. Pat. No. 6,086,430 describes an example of a connector that includes a means for allowing a host device to identify a type of connector. The specification describes an enhanced USB interface that provides for communication of standard USB signaling and non-standard USB signaling. The chassis within the host device (i.e., a data processing device) for receiving the enhanced USB connector has an enhanced USB receptacle providing a configuration so that a standard USB connector portion is not able to be mated with a non-standard USB portion.

U.S. Pat. No. 7,155,545 describes a second example of a connector that includes a means for allowing a host device to identify a type of connector. A standard USB connector has four pins such that a first pin connects to a power supply, a second pin transmits signal D+, a third pin transmits signal D−, and a fourth pin connects to ground. The hybrid USB connector of U.S. Pat. No. 7,155,545 has a shorter third pin for the D− signal. The shorter third pin ensures that the second pin connects to a complementary second pin of a host device connector before the shorter third pin connects to a complementary third pin of the host device connector. The host device recognizes the USB device correctly when host USB connector connects the USB device to the host.

U.S. Pat. No. 7,225,809 describes a removable data storage device that can include set-up information/data or the like, which information can be used by the host device into which the removable data storage device is connected to the host device. The removable data storage device and the host unit into which the removable data storage device is inserted can also include a mutual electronic keying arrangement to identify that a correct removable data storage device has been inserted.

U.S. 2007/023044 describes a therapy device for providing CPAP or the like. The therapy device includes an audio unit that can play an alarm or music (e.g., from a CD or the like).

U.S. 2008/0204992 describes a keyed interface between a USB stick and a host computer.

In the present specification where reference has been made to patent specifications, other external documents, or other sources of information, this is generally for providing a context for discussing certain features of embodiments of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

It is an object of certain embodiments of the invention is to provide an improved interfacing arrangement between a removable peripheral device and a host device, or to at least provide the public or the industry with a useful choice.

In a first aspect, the present invention broadly consists in a removable peripheral device and a breathing assistance unit, the breathing assistance unit in use forming part of a system for providing heated humidified air at a positive pressure to a user or patient, said removable peripheral device and said breathing assistance unit mutually adapted to be removably connectable to and in electrical communication with each other via a connection formed when said removable peripheral device is connected to said breathing assistance unit, said removable peripheral device comprising a first connector and a housing, said first connector external to said housing and extending from an end of said housing, said housing having a larger cross-section than said first connector, said breathing assistance unit comprising at least an exterior casing adapted to enclose at least electronic circuitry, and a second connector mounted within said exterior casing and accessible from outside said exterior casing, in use said first connector and said second connector removably connecting together to form said connection, said exterior casing further comprising an interfacing portion, said interfacing portion, said second connector and said housing formed so that in use at least a portion of said first connector housing adjacent said first connector end must interface with said interfacing portion in order to connect said removable peripheral device to said breathing assistance unit to make said connection.

Preferably, said removable peripheral device is a removable memory device of the type that has a read writable electronic memory for storing information electronically.

Preferably, the first connector portion is either a standard male electrical connector or a standard female electrical connector, and the second connector portion is one of a corresponding standard female electrical connector or a corresponding standard male electrical connector. Preferably said first connector portion is a standard male connector and said second connector portion is a corresponding standard female connector.

Preferably, said standard male electrical connector and said standard female electrical connector are a type of Universal Serial Bus connector.

Preferably, the standard male electrical connector and the standard female electrical connector are a type of Ethernet connector such as a RJ 45 connector.

Preferably said interfacing portion of said host device is a port or recess in an exterior surface of said exterior casing, said second connector portion mounted within said exterior casing a set distance from said exterior surface so that said at least one surface of said exterior housing must enter said port or recess in order for said first connector portion to removably connect to said second connector portion in electrical communication, said port or recess shaped such that a different removable peripheral device must have an end outline that fits within said port or recess in order to connect with said second connector portion.

Preferably, said interfacing portion and said housing are shaped so that said removable peripheral device can be removably connected in electrical communication with said host device in one orientation only.

Preferably said at least one surface of said exterior housing must pass at least 40 mm into said interfacing portion to make the connection.

Preferably, said exterior housing includes an indicator that provides an indication that said removable peripheral device has been correctly connected in removable electrical connection to said host device.

Preferably said indicator is a first marker on said housing, and said host device includes a corresponding second marker, said first and second markers adapted to align when said connection is made.

Preferably said host device is a breathing assistance unit, the breathing assistance unit in use forming part of a system for providing heated humidified air at a positive pressure to a user or patient.

Preferably said removable peripheral device includes an electronic memory adapted to electronically store data relating to a patient treatment regime, in use said data being used by said electronic circuitry of said host device so that said host device can provide said patient treatment regime to a patient.

Preferably, said data is set-up parameters for initially setting up said host device.

Preferably, said data is gathered from the use of the breathing assistance unit as part of a breathing circuit, said data being useful for review by a health professional for assessing a patient's use of said breathing assistance unit.

Preferably said host device includes an audio speaker and said removable peripheral device includes an electronic memory adapted to electronically store at least one audio file, in use said at least one audio file being used by said electronic circuitry of said host device so that said host device can play said at least one audio file.

Preferably said electronic memory of said removable peripheral device is partitioned into at least a first partition and a second partition, said first partition dedicated to at least a first function, and said second partition dedicated to at least a second function.

Preferably, said first partition is configured to store data relating to a patient treatment regime.

Preferably when the removable peripheral device is connected to the host device, the host device electronic circuitry is configured to in use automatically use the data relating to a patient treatment regime for operation of the host device, and said electronic circuitry of said host device is configured to use default settings when said removable peripheral device is not connected to the host device.

Preferably when the removable peripheral device is connected to the host device, the host device electronic circuitry is configured to in use automatically use the data relating to a patient treatment regime for operation of the host device, and said electronic circuitry of said host device is configured to in use require operational data to be entered by a user for operation of the host device when said removable peripheral device is not connected to the host device.

Preferably said first partition is electronically configured so that said host device can write to and read from said first partition, said first partition further configured so that a different host device cannot access said first partition.

Preferably, said second partition is electronically configured so that a different host device can write to and read from said second partition.

Preferably, said electronic memory of said removable peripheral device is electronically keyed to the electronic circuitry of said host device.

Preferably once one of said removable periphery device and a different removable peripheral device is electrically connected to said host device, said host device electronic circuitry is configured to automatically carry out an interrogation routine to determine whether said removable peripheral device or said different removable periphery device includes a correct electronic key.

Preferably said host device includes a user interface, said host device electronic circuitry configured to cause said user interface to display an error message if said interrogation routine determines that a removable periphery device electrically connected to said host device does not have said electronic key, said error message comprising a visual indication on said user interface or an error sound signal, or both.

Preferably said host device operates with limited or part functionality if a removable peripheral device electrically connected to the host device does not include said electronic key, or if no removable peripheral device is connected to the host device.

Preferably said host device includes a user interface in communication with said electronic circuitry and said host device electronic circuitry includes an integrated clock, said electronic circuitry and said user interface configured so that a user can set alarm times via said user interface, said host device adapted to play at least one said audio file as an alarm sound.

Preferably said removable peripheral device is adapted to electronically store at least two audio files, said host device having at least one alarm play mode settable by said user, a first mode in which said host device is adapted to play said at least two audio files sequentially, and a second mode in which the host device is adapted to play said at least two audio files in a random order.

Preferably said host device is adapted to sound a ‘buzzer’ type alarm if there are no audio files electronically stored on the removable peripheral device, or if there is no removable peripheral device connected in electrical communication with said host device.

Preferably, said audio files are stored in the second partition. In a second aspect the present invention broadly consists in a removable peripheral device and a host device, said removable peripheral device and said host device mutually adapted to be removably connectable to and in electrical communication with each other via a connection formed when said removable peripheral device is connected to said host device, said removable peripheral device comprising a first connector portion and an exterior housing, said first connector portion external to said exterior housing, said host device comprising at least an exterior casing adapted to enclose at least electronic circuitry that forms part of said host device, and a second connector portion mounted within said exterior casing and accessible from outside said exterior casing, in use said first connector portion and said second connector portion removably connecting together to form said connection, said host device further including an interfacing portion, said interfacing portion and said exterior housing adapted so that at least one surface of said exterior housing of said removable peripheral device must interface with said interfacing portion when connecting said removable peripheral device to said host device to make said connection, said interfacing portion shaped such that a different removable peripheral device must have an end outline that fits within said interfacing portion in order to connect with said second connector portion, said interfacing portion and said exterior housing being shaped so that said removable peripheral device can be removably connected in electrical communication with said host device in one orientation only.

The second aspect of the present invention may comprise any one or more of the features mentioned in respect of the first aspect of the present invention.

The term “comprising” as used in this specification means “consisting at least in part of. When interpreting each statement in this specification that includes the term “comprising” features other than that or those prefaced by the term may also be present. Related terms such as “comprise” and “comprises” are to be interpreted in the same manner.

To those skilled in the art to which the invention relates, many changes in construction and widely differing embodiments and applications of the invention will suggest themselves without departing from die scope of the invention as defined in the appended claims. The disclosures and the descriptions herein are purely illustrative and are not intended to be in any sense limiting.

The invention consists in the foregoing and also envisages constructions of which the following gives examples only.

### BRIEF DESCRIPTION OF THE DRAWINGS

Preferred embodiments of the present invention will be described by way of example only and with reference to the accompanying drawings.

FIG. 1 shows a preferred embodiment of a peripheral device and a host device that are arranged and configured in accordance with certain features, aspects and advantages of the present invention with the peripheral device shown removed or unconnected from a port or recess in the host device.

FIG. 2a shows the peripheral device and the host device of FIG. 1 with the peripheral device connected to the host device and the devices in electrical communication with one another.

FIG. 2b shows an alternative configuration of the host device and peripheral device of the present invention, with the peripheral device connected to the host device and in electrical communication with the host device.

FIG. 3a shows an end view of the peripheral device of the preferred embodiment of the present invention viewed in the direction of arrow A of FIG. 1.

FIGS. 3b and 3c show end views of alternative forms of the removable peripheral device of the present invention viewed in the direction of arrow A of FIG. 1.

FIGS. 4a and 5a show perspective views of alternative embodiments of the peripheral device and the host device of the present invention, wherein the peripheral devices are shown removed or unconnected to the host devices.

FIG. 4b shows a side view of a portion of the host device of FIG. 4a with a recess of a connection interface of the host device viewed in the direction of arrow B of FIG. 4a.

FIG. 5b shows a side view of a portion of the host device of FIG. 5a with a recess of a connection interface of the host device viewed in the direction of arrow C of FIG. 5a.

FIG. 6 shows a cross sectional view of the preferred embodiment of the peripheral device and the host device of FIG. 1 viewed from a front of the host device and showing the removable peripheral device connected to the host device.

FIG. 7a is a schematic representation of the peripheral device and the host device of the preferred embodiment of the present invention, which representation schematically shows electrical connections between the host device and the peripheral device with the peripheral device shown schematically as having a partitioned memory.

FIG. 7b is a schematic representation of the peripheral device of the present invention, which representation shows the peripheral device with a partitioned memory electrically connected to a host device other than the host device of the present invention.

### DETAILED DESCRIPTION OF THE PREFERRED EMBODIMENT

The present invention generally relates to an electrical communication connection arrangement between a removable peripheral device 1 and a host device 2. The peripheral device 1 may be any equipment removably connected directly to the host device, such as a cable with an integral connector that can be removably connected for electrical communication to the host device, a second device or another piece of equipment. In the preferred embodiment, the removable peripheral device is a removable memory device 1 that has a read-write memory and an integral connector 7. In the most preferred embodiment, the removable memory device 1 is a Universal Serial Bus (USB) memory stick-type of electronic storage device used for storing information electronically. Alternatively, the removable peripheral device may be any type of removable, portable, pocket-sized electronic storage device, such as a smart card, for example but without limitation. The removable memory device 1 may also include embedded integrated circuits that can process information and store data.

The integral connector 7 of the illustrated removable peripheral device 1 is adapted for removable connection to the host device 2 such that, in use, the connection forms an electrical communication pathway.

In one embodiment, the host device 2 is an integrated assisted breathing and humidification unit of the type that provides heated humidified air at a positive pressure to a user or patient for the treatment of ailments such as sleep apnoea or snoring. An example of an integrated unit of a similar type is described in PCT application WO2008/056993. Alternatively, the host device may be a blower unit, or a humidification unit. Alternatively, the host device may be any device that contains electronic processing equipment with which the removable peripheral device 1 communicates when connected in electrical communication.

The electronic processing equipment typically includes electronic control circuitry to control the electrical functions of the host device 2. The host device 2 preferably includes power circuitry for powering the control circuit using electrical power supplied by an external power source to the host device, such as an appliance power cord of the type typically found in a domestic environment.

The invention now will be described in relation to the preferred embodiment where the removable peripheral device 1 is a USB memory stick and the host device 2 is an integrated assisted breathing and humidification unit.

The USB memory stick 1 of the present invention includes an external housing 11. The housing 11 may be manufactured in at least two pieces that are assembled together to enclose internal electronic components of the USB memory stick, for example. Alternatively, the body or housing 11 can be molded to encapsulate the internal electronic components or the body or housing 11 can be formed in any other suitable manner. The integral connector 7 preferably is external to the housing 11 at one end of the housing 11 so that the integral connector 7 is easily accessible.

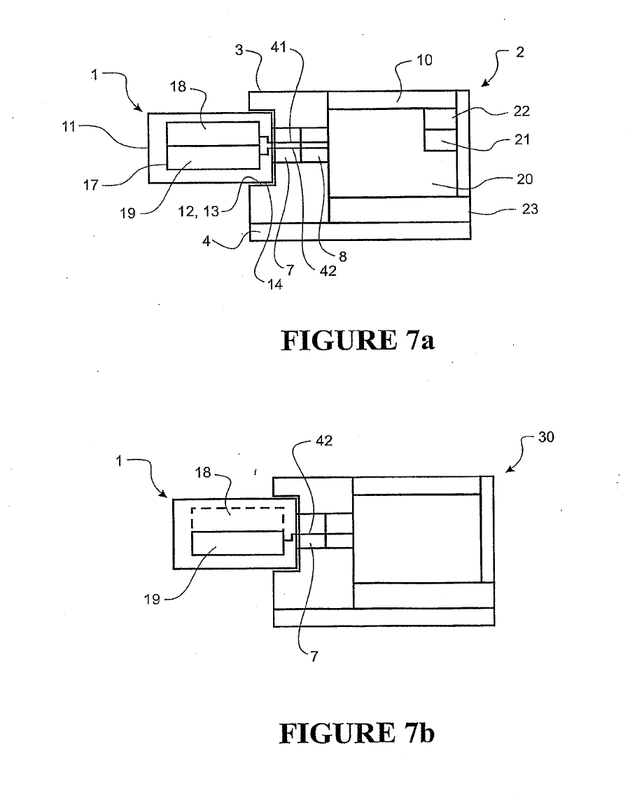

The internal components of the integrated unit 2, including the control circuitry 20 and power supply circuitry 23 (shown schematically in FIGS. 7a and 7b), preferably are contained within an outer casing 3. The casing 3 typically is manufactured in at least two pieces that are assembled together to enclose or encase the internal components of the integrated unit 2. Alternatively, a single piece casing 3 can be used that fits over the integrated unit's internal components. The single piece casing can attach to a base 4 of the integrated unit with the base 4 being separate to the casing 3.

The integrated unit 2 preferably includes a user interface 10. The user interface 10 can include input controls such as, for example, dials, knobs, touch screens and other suitable input devices. The user interface 10 may further include output devices, such as a display screen and audio output via a speaker.

For ease of explanation, when referring to the casing 3 in this specification, the casing 3 should be taken to include any part of the integrated unit 2 that encloses the internal components of the host device 2. For example, part of the user interface 10, such as a control knob 5 or display screen 6, could be said to form part of the host device casing 3. Alternatively, part of the base 4 could be said to form part of the integrated unit casing 3.

The USB memory stick 1 communicates electrically with the integrated unit 2 via a connection that is made or formed when the memory stick 1 and the integrated unit 2 are brought together or connected in use. The connection is formed from the first connector portion 7 and a second connector portion 8. As discussed above, the first connector portion 7 is integral to the USB memory stick 1. The second connector portion 8 can be integral to the integrated unit 2. In use, the first connector portion 7 and the second connector portion 8 are removably connected together to make the connection between the USB memory stick 1 and the integrated unit 2.

In the preferred form, the first connector portion 7, which is part of the removable USB memory stick connector, is a male connector and the second connector portion 8, which is part of the integrated unit 2, is a corresponding female connector. However, these could be reversed in alternative forms with the first connector portion 7 being a female connector and the second connector portion 8 being a corresponding male connector.

In the preferred embodiment the first and second connector portions 7, 8 are standard connectors. Preferably, the standard connector of the preferred embodiment is a Universal Serial Bus (USB) connector. For example, the USB connector could be any one of: a Standard-A plug and receptacle, a Standard-B plug and receptacle, a Mini-A plug and receptacle, a Mini-B plug and receptacle, or any other USB standard connector. Alternatively, the removable peripheral device may include an alternative standard connector, such as a RJ45 Ethernet standard connector.

### Keyed Removable Memory Device to Host Device Interface

For certain host devices, it is desirable to physically key the first connector portion 7 to the second connector portion 8 so that a particular type of peripheral device or series of peripheral devices, such as memory card 1, are preferred for use in combination with the host device, such as integrated unit 2. The present invention achieves a keyed arrangement between the USB memory stick 1 and the integrated unit 2 by having an interfacing portion on the integrated unit 2 that interfaces with at least one surface of the USB memory stick housing 11 when connecting the USB memory stick 1 to the integrated unit 2.

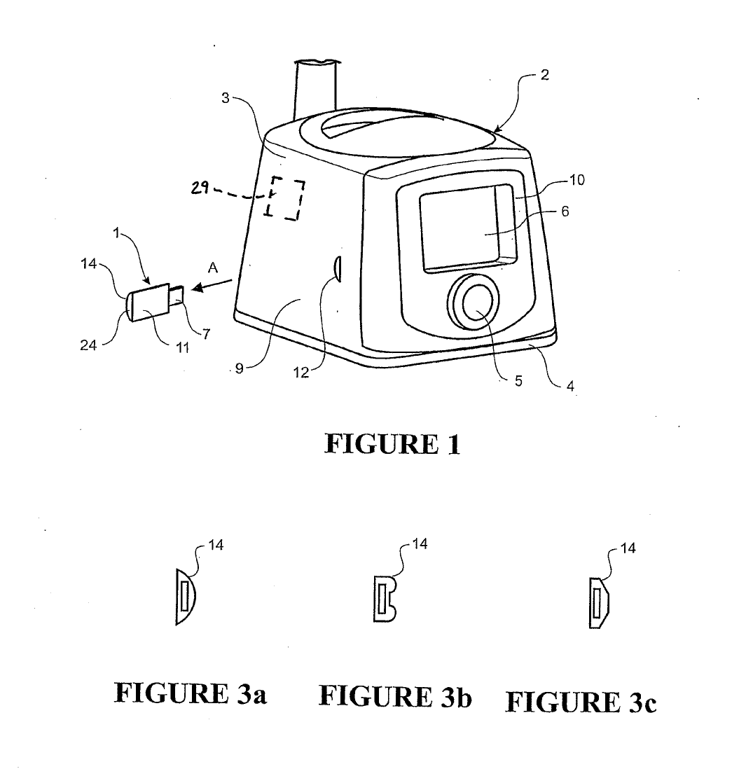

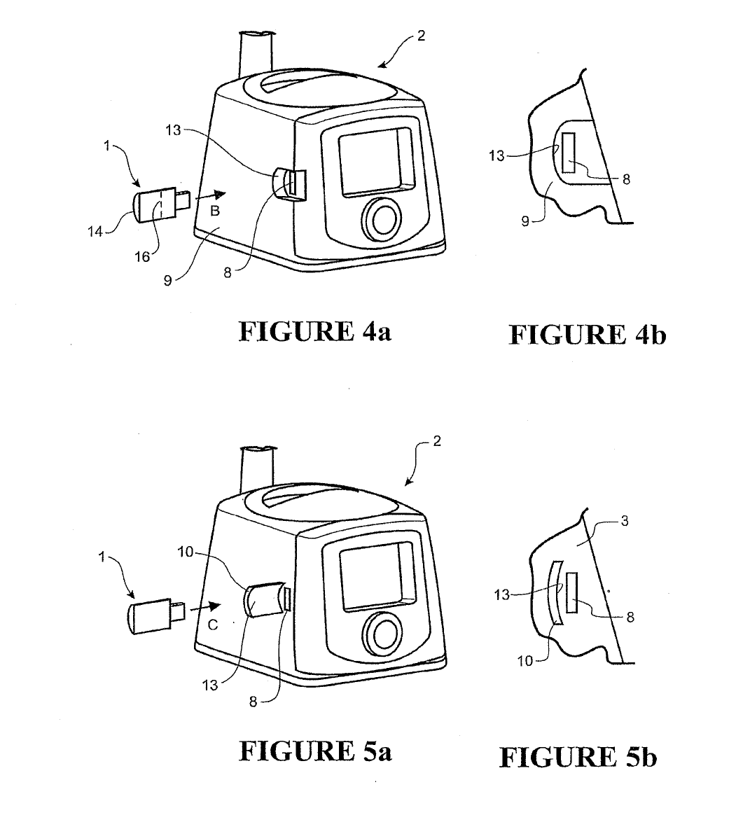

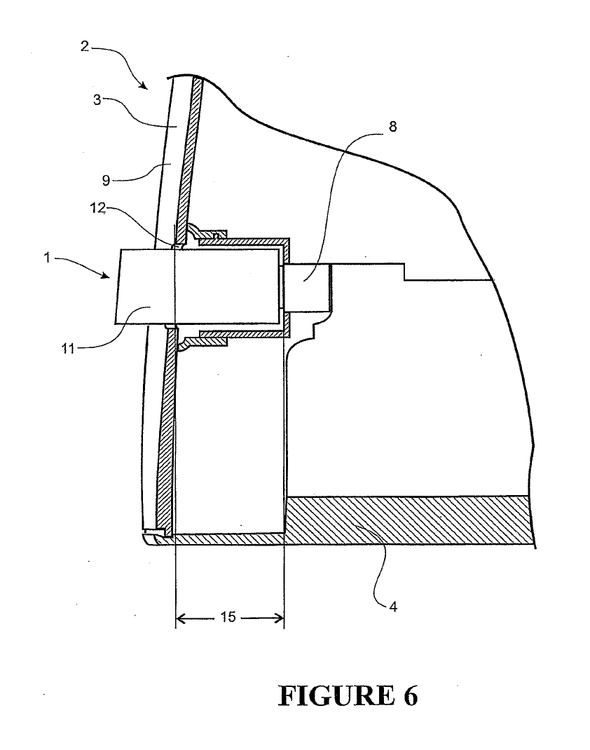

In the preferred embodiment, the interfacing arrangement of the present invention is achieved by locating the second connector portion 8 within the integrated unit casing 3 a set distance 15 (as shown in FIG. 6) from an exterior surface 9 or edge 10 of the integrated unit casing 3. In order to connect to the host device 2, at least a portion of the USB memory stick body 11 can be located between the exterior face 9 or edge 10 and the second connector portion 8 to mate connector halves 7, 8 together. In the most preferred embodiment of FIG. 1, when the USB memory stick 1 is connected to the integrated unit 2, an interfacing portion 12 of the integrated unit 2, which is located at the exterior face 9, lies adjacent to a corresponding keying portion 14 of the USB memory stick housing 11 such that the interfacing portion 12 and the keying portion 14 interface in a keyed arrangement. In the variants of FIG. 4a or 5a, with the USB memory stick 1 connected to the integrated unit 2, an interfacing surface 13 of the integrated unit 2, which can be located between the exterior face 9 or edge 10 and the second connector portion 8, lies adjacent to the corresponding keying portion 14 of the USB memory stick housing 11 such that the interfacing surface 13 and the keying portion 14 interface in a keyed arrangement.

For example, the interfacing portion 12 may act as a keyway and the keying portion 14 may act as a key. Alternatively, the interfacing portion 12 may act as a key and the keying portion 14 may act as a keyway.

In the preferred embodiment of the present invention as shown in FIG. 1, the interfacing portion 12 can comprise an opening, such as a port or recess, for example, that is formed through an exterior face 9 of the integrated unit 2. The port or recess 12 is shaped such that a different peripheral device, such as a standard common USB memory stick, must have an end outline (i.e., the outer perimeter or outline when viewed from one end) that fits within said port or recess in order to connect with the second connector portion. Typically, a standard memory stick will not fit within the port or recess 12 due to interference between the body of the standard memory stick and the port or recess 12.

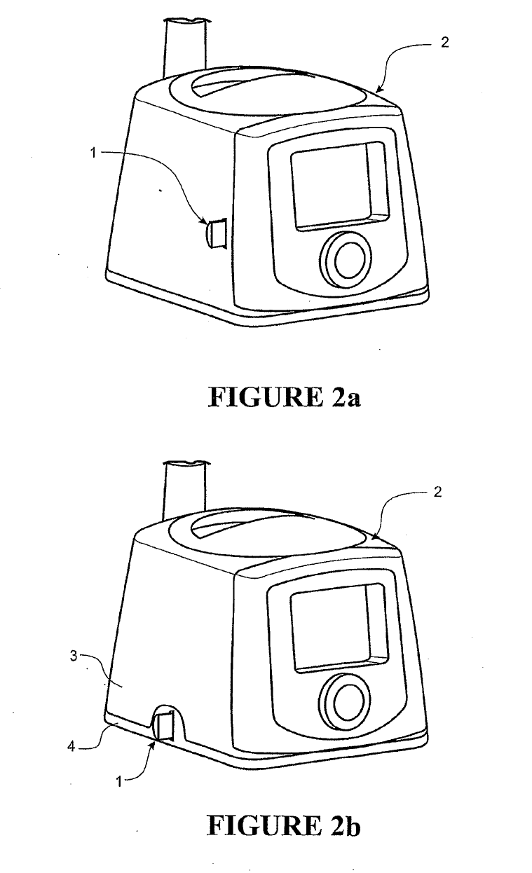

FIG. 2a shows the USB memory stick 1 installed or connected to the host device 2. In FIG. 2b, the USB device interfaces with the host device via the base 4 of the outer casing 3 of the host device 2.

Alternatively, the interfacing portion may be formed as an interfacing surface 13 extending between the exterior surface 9, or edge 10 of the integrated unit 2, as shown in FIGS. 4a and 5a. The interfacing surface 13 can be located adjacent to the longitudinal axis of the second connector 8 so that the keying portion 14 of the USB memory stick housing 11 interfaces with the interfacing surface 13 to allow electrical connection of the USB memory stick 1 and the integrated unit 2.

For the embodiments shown in FIGS. 2a and 2b, and for the embodiments shown in FIGS. 4 and 5, the port or recess 12 (FIGS. 2a and 2b) and the interfacing surface 13 (FIGS. 4 and 5) are shaped such that a different peripheral device, such as a standard common USB memory stick, must have an end outline (i.e., the outer perimeter or outline when viewed from one end) that fits into the port or recess 12 or alongside the interfacing surface 13 without interfering with, or without an interference fit with, the port or recess 12 or the interfacing surface 13 in order to suitably connect with the second connector portion 8. In such standard devices that have an end outline that will not fit within the interfacing surface cannot be connected.

In the preferred embodiment, the keying portion 14 and the interfacing portion 12 or the interfacing surface 13 are mutually shaped so that the USB memory stick 1 may be connected in electrical communication with the integrated unit 2 in one orientation only. The mutual shaping assists the user to correctly connect the USB memory stick 1 to the integrated unit 2. A preferred shape for the housing of the removable peripheral device is shown in FIG. 3a. Examples of alternative possible peripheral device housing shapes that provide for a single orientation connection are shown in FIGS. 3b and 3c. Any other shape may be used wherein the shape allows only one mating orientation within 360 degrees of rotation about a longitudinal axis of the removable memory device 1.

The present invention provides the benefit that a keying arrangement can be achieved for the connection between the peripheral device and the host device without the need to develop proprietary connectors or the need to use a less common standard connector configuration. This benefit results in simpler and lower cost manufacturing.

Alternatively, the present invention may use a proprietary connector. However, the benefits of such an embodiment of the present invention would not be as great as the benefits of the preferred embodiment of the present invention. A benefit of using a proprietary connector with the present invention is that no keying details need to be considered for the connector itself. The keying arrangement is provided for in the shape of the host device interfacing portion 12 or the interfacing surface 13 and the corresponding shape of the keying portion 14 of the removable peripheral device housing 11.

As shown in the figures, the keying portion 14 of the removable peripheral device extends along one side of the body from end to end. However, the keying portion 14 may extend along a side of the body for a portion of the side only. For example, the keying portion 14 can extend only beyond the interfacing portion when the removable peripheral device 1 is installed or connected to the host device 2. The keying portion may not extend to the end distal from the connector 7.

In order to implement the preferred embodiment of present invention, the housing 11 of the removable peripheral device 1 may be larger than necessary for the normal electronic functioning of the removable peripheral device 1. For example, modern removable memory devices have become relatively slim and it is possible that a different removable memory device that has a slim body may be connectable to the host device 2, wherein the different removable memory device body does not provide an interface portion adjacent the host device interfacing portion. That is, a different, smaller removable memory device may be small enough to fit into, for example, the port or recess formed by the interfacing portion 12 in the casing 3 of the host device 2 of the preferred embodiment. This means that the keying arrangement between the removable memory device housing and the host device casing may not always prevent non-keyed removable memory devices from being electrically connected to the host device. It can be seen that the different, smaller, removable memory device must have an end outline (i.e., the outer perimeter or outline when viewed from one end) that will fit inside, or that will fit within, the interfacing portion 12 or interfacing surface 13 without interfering with, or without an interference fit with, the portion 12 or surface 13, and that will also allow the connector portion of the different device to connect to the second connector portion 8.

Because the second connector portion 8 is located a set distance 15 from the exterior surface 9 or edge 10, the peripheral device or removable memory device 2 should be a certain length to extend from the second connector portion 8 beyond the exterior surface 9 or edge 10 to allow a user to easily grasp the peripheral device 1 for removal from the host device 2. The extension is a particularly useful feature of the preferred embodiment, wherein the USB memory stick 1 interfaces with the integrated unit 2 via a port or recess in the integrated unit casing 3 that forms the interfacing portion 12. By locating the second connector portion 8 a particular depth or set distance 15 (as shown in FIG. 6) from the exterior surface 9 in which the port or recess 12 is formed, the USB memory stick 1 should be a certain length to be able to be connectable to and removable from the integrated unit 2. A different removable peripheral device may have a housing small enough to enter the interfacing portion 12; however, it may be too short to be able to be connected to the integrated unit 2. In the preferred embodiment, the second connector is about 5 mm to about 60 mm from the exterior surface 9 or edge 10 of the integrated unit 2. Preferably, the second connector is about 40 mm from the exterior surface or edge. The housing 11 therefore passes about 40 mm into the interfacing portion to make the connection. In some embodiments, the removable peripheral device is correctly connected in removeable electrical connection to the breathing assistance unit when the removable peripheral device is fully inside or has an outermost surface that is generally flush with an exterior surface of the housing of the breathing assistance apparatus.

In the most preferred embodiment, an end 24 of the USB memory stick protrudes a few millimeters from the surface 9 or edge 10 when electrically connected to the integrated unit. The USB memory stick and the host device (i.e., integrated unit 2) can be locked together if required. To remove the USB memory stick, end 24 of the memory stick 1 initially can be pushed in a direction into the integrated unit 2. Pushing the memory stick 1 in this direction activates a push release mechanism in the integrated unit 2 that pops the USB memory stick out of the integrated unit 2. When the memory stick 1 is released, a user can easily grasp the protruding part of the memory stick 1 for removal.

The USB memory stick in the preferred embodiment has an indicator 16 to provide to the user an indication when the USB memory stick 1 has been correctly connected to, or has been fully engaged with, the integrated unit 2. The indicator in the preferred embodiment is a line 16 or first marker 16 on the housing 11 of the USB memory stick 1. The line or first marker 16 may align with a corresponding portion of the integrated unit casing 3 or a corresponding line or second marker on the casing 3 to indicate that the USB memory stick 1 is fully engaged with the integrated unit 2.

### Electronic Keying

As described above, it may be possible for a different removable peripheral device with a slim body to interface with the integrated unit 2 of the preferred embodiment without the body of the different removable peripheral device specifically interfacing with the integrated unit interfacing portion 12 or interfacing surface 13.

However, in the preferred embodiment of the present invention, the USB memory stick 1 also includes electronic keying. Once one of the removable USB memory stick of the present invention and a different removable periphery device is electrically connected to the integrated unit 2 of the preferred embodiment, the integrated unit control circuitry contained within the integrated unit 2 automatically carries out an interrogation routine to determine whether the USB memory stick or the different removable periphery device has a correct electronic key.

The interrogation routine may comprise the control circuitry reading the USB vendor or USB product ID or both from the USB memory stick and comparing this to a predefined vendor or vendor ID or both. Alternatively, the interrogation routine may comprise the control circuitry reading a predefined ID string from the USB memory stick and comparing this to an expected ID string. Alternatively, the interrogation routine may comprise the control circuitry reading any combination of the USB vendor, the USB product ID and an ID string and comparing this information to predetermined values.

Should the interrogation routine determine that the removable peripheral device connected to the host device is not an intended or allowable device, the integrated unit control circuitry 20 is configured to cause the host device user interface 10 to display an error message. The error message comprises a visual indication on the user interface 10, an error sound signal or both. Operation or part functionality of the integrated unit 2 may be limited while a different (i.e., non-recognized or non-approved) removable peripheral device is electrically connected to the integrated unit 2, if no removable peripheral device is connected to the integrated unit, or both.

### Electronic Memory Configuration

In the preferred embodiment of the present invention, the USB memory stick that forms the removable memory device 1 contains user or patient data or information relating to a user or patient treatment regime, this data being pre-loaded or pre-configured for an individual patient. The data is used to control the integrated unit 2 of the preferred embodiment to provide the patient treatment regime or to provide set-up parameters for initially setting up the integrated unit 2 to provide the patient treatment regime to the user or patient. Alternatively, data is gathered from the use of the integrated unit 2 as part of a breathing circuit, which data may be useful for review by a health professional. For example, the data can include compliance data or the like and can be stored on the removable memory device 1.

In operation, when the USB memory stick 1 is connected to the integrated unit 2, the integrated unit 2 is configured to automatically use the patient related data stored in the memory of the USB memory stick 1. If the USB memory stick 1 is not connected to the integrated unit 2, or if a different removable peripheral device is connected to the integrated unit 2, the integrated unit 2 either will use default settings residing in the integrated unit's control circuitry 20 or will require operational data to be directly entered by a user or the patient. In the most preferred form, the memory of the USB memory stick 1 is configured to also allow storage of other data files, such as audio files for example.

In the preferred embodiment, the memory capacity 17 of the USB memory stick 1 is partitioned as indicated schematically in FIG. 7a. At least a first partition 18 of the total memory capacity of the USB memory stick is dedicated to at least a first function and at least a second partition 19 of the USB memory stick memory is dedicated to at least a second function.

In the preferred embodiment, the first partition 18 is configured to contain user or patient data or information relating to a user or patient treatment regime as outlined above. The data in the first partition 18 is used to control the integrated unit 2 to provide the patient treatment regime, the data in the first partition 18 contains set-up parameters for initially setting up the integrated unit 2 to provide the patient treatment regime to the user or patient, or the data in the first partition 18 is data gathered from the use of the integrated unit 2 as part of a breathing circuit, which data may be useful for review by a health professional (e.g., compliance data or similar).

In operation, when the USB memory stick 1 is connected to the integrated unit 2, the integrated unit 2 is configured to automatically use the patient related data stored in the first partition 18 as indicated by line 41 in FIG. 7a. If the USB memory stick 1 is not connected to the integrated unit 2, or if a different removable peripheral device is connected to the integrated unit 2, the integrated unit 2 either will use default settings residing in the integrated unit's control circuitry or operational data will be requested to be entered by a user or the patient.

In the preferred embodiment, the first partition 18 is electronically configured to be written to and read by the integrated unit 2 of the present invention only as indicated by line 41 in FIG. 7a. If the USB memory stick 1 of the present invention is installed in a host device 30 other than the integrated unit 2 of the present invention, the host device 30 being, for example, a user's home personal computer, the first partition 18 preferably will not be accessible by the other host device 30, as indicated schematically in FIG. 7b. In the preferred embodiment of the present invention, the first partition 18 contains data only accessible by the host device 2 of the present invention or by another device that has been correctly set up to access the data, such as a hospital computer or the like.

Preferably, the second partition 19 is configured to allow storage of other data files that can be chosen by the user or patient, such as audio files, for example. When installed in a host device 30 other than the integrated unit 2, the second partition 19 is accessible by the other host device 30 (e.g., a user's home personal computer). The second partition 19 is accessible by the host device 2 and other host devices 30, as indicated by line 42 in FIGS. 7a and 7b. The USB memory stick 1 interfaces in a normal manner to another host device 30 that is configured to accept USB type devices with a limitation that the first partition 18 generally is not accessible. A user may save and delete electronic data to and from the second partition 19 of the USB memory stick 1 when the memory stick 1 is connected to a host device other than the host device 2 (e.g., treatment apparatus), as indicated schematically by line 42 in FIG. 7b.

### Alarm Functionality

In the preferred embodiment, the integrated unit's control circuitry 20 includes an integrated time clock 21 and a user settable alarm function 22 whereby a user or patient can set alarms via the user interface 10. For example, the host device control circuitry and user interface can be adapted to play at least one audio file as an alarm sound based on a set alarm time. Accordingly, the unit 2 preferably comprises at least one speaker 29 or another suitable line out line for connection to a remotely positioned speaker.

In the preferred embodiment, with the USB memory stick 1 connected to the integrated unit 2, the alarm function of the integrated unit's control circuitry 20 can play an audio file as an alarm sound when a user set alarm is activated by the integrated unit's control circuitry 20. In the preferred embodiment, the audio file that is played has been stored in the second partition 19 of the USB memory stick 1.

In the preferred embodiment, the integrated unit 2 has at least one alarm play mode. In a first alarm play mode, the integrated unit 2 is adapted to play at least one audio file stored on the USB memory stick 1. If desired, the file can be played repeatedly until the user turns the alarm off. In a second alarm play mode, at least two audio files can be played sequentially for sequentially activated alarms. In a third alarm play mode, the integrated unit can play at least two music files stored on the USB memory stick 1 in a random order for sequentially activated alarms. In a fourth alarm play mode, the integrated unit is adapted to allow a user to manually select a particular music file to be played for a particular alarm condition or conditions.

In the preferred embodiment, should the USB memory stick 1 not be connected to the integrated unit 2, or if a different removable peripheral device is connected to the integrated unit 2, the integrated unit 2 will sound a default alarm, such as a buzzer-type alarm, for example but without limitation. Alternatively, if the USB memory stick 1 of the present invention is installed in the integrated unit 2, but the USB memory stick 1 does not contain any audio files, then the integrated unit 2 can sound the buzzer-type alarm, for example or without limitation.

The foregoing description of the invention includes preferred forms thereof. Modifications may be made thereto without departing from the scope of the invention.

What is claimed is:

1. A removable peripheral device and a breathing assistance unit, the breathing assistance unit in use forming part of a system for providing heated humidified air at a positive pressure to a user, said removable peripheral device and said breathing assistance unit mutually adapted to be removably connectable to and in electrical communication with each other via a connection formed when said removable peripheral device is connected to said breathing assistance unit;
said removable peripheral device comprising a first connector and a removable peripheral device housing, said first connector being external to said removable peripheral device housing, said first connector extending from an end of said removable peripheral device housing, said removable peripheral device housing having at least a portion that comprises a larger cross-section than said first connector;
said breathing assistance unit comprising a breathing assistance unit exterior casing adapted to enclose electronic circuitry, a second connector being mounted within said breathing assistance unit exterior casing, said breathing assistance unit exterior casing comprising an opening and said second connector being accessible from outside said breathing assistance unit exterior casing;
in use said first connector and said second connector removably connecting together to form said connection;
said breathing assistance unit exterior casing comprising an interfacing portion, said interfacing portion, said second connector and said removable peripheral device housing formed so that in use at least a portion of said removable peripheral device housing adjacent said first connector end interfaces with said interfacing portion of said breathing assistance unit exterior casing in order to connect said first connector of said removable peripheral device to second connector of said breathing assistance unit.

2. The removable peripheral device and the breathing assistance unit as claimed in claim 1, wherein said removable peripheral device comprises a removable memory device of the type that has a read write electronic memory for storing information electronically.

3. The removable peripheral device and the breathing assistance unit as claimed in claim 2, wherein said read write electronic memory of said removable peripheral device is partitioned into at least a first partition and a second partition, said first partition being dedicated to at least a first function and said second partition being dedicated to at least a second function, said first partition being configured to store data relating to a patient treatment regime and said first partition being electronically configured so that said breathing assistance unit can write to and read from said first partition, said first partition further configured so that a different breathing assistance unit cannot access the first partition and said second partition being electronically configured so that a host device can write to and read from said second partition.

4. The removable peripheral device and the breathing assistance unit as claimed in claim 1, wherein said removable peripheral device includes an electronic memory adapted to electronically store data relating to a treatment regime, in use said data being used by electronic circuitry of said breathing assistance unit so that said breathing assistance unit can provide said treatment regime.

5. The removable peripheral device and the breathing assistance unit as claimed in claim 4, wherein when said removable peripheral device is connected to said breathing assistance unit, electronic circuitry of said breathing assistance unit is configured to in use automatically use data relating to said treatment regime for operation of said breathing assistance unit, and said electronic circuitry of said breathing assistance unit is configured to use default settings when said removable peripheral device is not connected to said breathing assistance unit.

6. The removable peripheral device and the breathing assistance unit as claimed in claim 4, wherein said data comprises set-up parameters for initially setting up said breathing assistance unit.

7. The removable peripheral device and breathing assistance unit as claimed in claim 4, wherein said data comprises use data gathered during use of said breathing assistance unit as part of a breathing circuit, said use data being useful for review by a health professional for assessing a use of said breathing assistance unit.

8. The removable peripheral device and the breathing assistance unit as claimed in claim 1, wherein
said interfacing portion of said breathing assistance unit exterior casing comprises a port or a recess in an exterior surface of said breathing assistance unit exterior casing, said second connector portion is mounted within said breathing assistance unit exterior casing a set distance from said exterior surface of said breathing assistance unit exterior casing such that at least one surface of said removable peripheral device housing enters said port or said recess in order for said first connector portion to removably connect to said second connector portion in electrical communication; and
said port or said recess shaped such that said removable peripheral device must have a first end housing end outline that fits within said port or said recess in order to connect with said second connector portion.

9. The removable peripheral device and the breathing assistance unit as claimed in claim 8, wherein at least about 40 mm of said removable peripheral device housing must pass into said interfacing portion of said breathing assistance unit exterior casing to make the connection and said at least one surface of said removable peripheral device housing is at least partially located along said at least about 40 mm of said removable peripheral device housing.

10. The removable peripheral device and the breathing assistance unit as claimed in claim 1, wherein said interfacing portion of said breathing assistance unit exterior casing and said removable peripheral device housing are shaped so that said removable peripheral device can be removably connected in electrical communication with said breathing assistance unit in one orientation only.

11. The removable peripheral device and the breathing assistance unit as claimed in claim 1, wherein said removable peripheral device is correctly connected in removable electrical connection to said breathing assistance unit when said removable peripheral device comprises an outermost surface that is generally flush with a surface of said breathing assistance unit exterior casing.

12. The removable peripheral device and the breathing assistance unit as claimed in claim 1, wherein said breathing assistance unit comprises an audio speaker and said removable peripheral device includes an electronic memory adapted to electronically store at least one audio file, in use said at least one audio file being used by electronic circuitry of said breathing assistance unit so that said breathing assistance unit can play said at least one audio file.

13. The removable peripheral device and the breathing assistance unit as claimed in claim 12, wherein said removable peripheral device comprises a removable memory device of the type that has a read write electronic memory for storing information electronically, said read write electronic memory of said removable peripheral device is partitioned into at least a first partition and a second partition, said audio files being stored in the second partition, in use said audio files being used by said electronic circuitry of said breathing assistance unit so that said breathing assistance unit can play said audio files and said first partition being configured so that a host device different from said breathing assistance unit cannot access the first partition while the second partition is configured so that a different host device can access the second partition.

14. The removable peripheral device and the breathing assistance unit as claimed in claim 1, wherein said electronic memory of said removable peripheral device is electronically keyed to electronic circuitry of said breathing assistance unit and
once one of said removable peripheral device and a different removable peripheral device is electrically connected to said breathing assistance unit, said breathing assistance unit electronic circuitry is configured to automatically carry out an interrogation routine to determine whether said removable peripheral device or said different removable periphery device includes a correct electronic key.
